(19) United States
(12) Patent Application Publication
(10) Pub. No.: US 2012/0158113 A1
(43) Pub. Date: Jun. 21, 2012

(54) Rectilinear Electrode Contact For Neural Stimulation

(75) Inventors: Claude Jolly, Innsbruck (AT); Anandhan Dhanasingh, Innsbruck (AT)

(73) Assignee: MED-EL ELEKTROMEDIZINISCHE GERAETE GMBH, Innsbruck (AT)

(21) Appl. No.: 13/325,213

(22) Filed: Dec. 14, 2011

Related U.S. Application Data
(60) Provisional application No. 61/423,369, filed on Dec. 15, 2010.

(51) Int. Cl. A61F 11/04; A61N 1/05

(52) U.S. Cl. 607/137

(57) ABSTRACT

A cochlear implant electrode is described. An elongate flexible electrode array has a linear central axis and an outer surface with electrode contacts for electrical stimulation of nearby neural tissue. At least one of the electrode contacts is a linear electrode contact forming an elongated rectilinear surface along a line parallel to the linear central axis of the electrode array.

This application claims priority from U.S. Provisional Patent Application 61/423,369, filed Dec. 15, 2010; incorporated herein by reference.

### TECHNICAL FIELD

The present invention relates to an implantable electrode for neural stimulation systems.

### BACKGROUND ART

The individual electrode contacts used in multi-channel neural stimulation electrodes tend to lose stimulation specificity due to current spread, especially when increased current intensity is needed to depolarize the nerve fibers. Adjacent electrode contacts also tend to stimulate overlapping neural populations, and as more current is applied to the surrounding tissue, more neurons in geometric space are recruited for firing. In an idealized isotropic conductive space in an infinite medium, current spreads uniformly in three dimensions. But current spreads in more complex ways in an anisotropic medium in real three dimensional space, depending on the resistance of the tissue near and around the electrode contacts.

In the past, channel interaction between adjacent electrode contacts or from a single electrode contact to a nerve population has been dealt with by increasing the interaction in a close local region while further reducing it from the electrode combination using simultaneous electrode contact stimulation with in-and out-of-phase firing patterns. Focusing current to a specific geometrical space at a certain time of stimulation has been addressed by using a combination of electrode contacts which may be in and/or out of phase and by multi-polar stimulation (e.g., tripolar or quadrupolar stimulation). Another approach has been the suggested use of a ring disk electrode contact forming an active and ground electrode complex which provides the same focusing action as with multi-polar stimulation but this also requires an independently activated ground electrode contact for each active electrode contact.

### SUMMARY

Embodiments of the present invention are directed to a multi-channel cochlear implant electrode. An elongate flexible electrode array has a linear central axis and an outer surface with electrode contacts for electrical stimulation of nearby neural tissue. At least one of the electrode contacts is a linear electrode contact forming an elongated rectilinear surface along a line parallel to the linear central axis of the electrode array.

In specific embodiments, there may be multiple linear electrode contacts and some of the linear electrode contacts may be distributed along different lines parallel to the linear central axis of the electrode array. One or more linear electrode contacts may be located towards the apical end of the electrode array. One or more linear electrode contacts may be located around a circumferential outer surface of the electrode array. At least one linear electrode contact may be located at an apical end outer surface of the electrode array.

At least a portion of the linear electrode contact may be elevated above the outer surface of the electrode array. For example, the elevated portion may be substantially centered within the at least one linear electrode contact. In addition or alternatively, at least a portion of the linear electrode contact may be recessed below the surface of electrode array.

A linear electrode contact may include a rounded center segment. Or there may be a rounded end segment, such as an arrangement where there is a rounded end segment at each end. A linear electrode contact may include multiple wave shape segments each following a line off the linear central axis arranged so that overall the linear electrode contact follows a line parallel to the linear central axis. A linear electrode contact may be positioned substantially the same distance from an apical end of the electrode array as another electrode contact.

### BRIEF DESCRIPTION OF THE DRAWINGS

FIG. 1 shows an example of a multi-channel implant electrode having linear electrode contacts according to an embodiment of the present invention.

FIG. 2 shows a conventional multi-channel cochlear implant electrode according to the prior art which is implanted in a human cochlea.

FIG. 3 shows a multi-channel cochlear implant electrode according to an embodiment of the present invention which is implanted in a human cochlea.

FIG. 4 A-C shows examples of specific linear electrode contact geometries according to specific embodiments of the present invention.

FIG. 5 A-B shows examples of a multi-channel implant electrode with linear electrode contacts having multiple wave shape segments according to another embodiment of the present invention.

FIG. 6 shows an example of a multi-channel implant electrode according to another embodiment of the present invention having different distributions of linear and conventional electrode contacts.

### DETAILED DESCRIPTION

It would be advantageous for electrical stimulation of neurons to develop electrodes and electrode contact geometries that can restrict the current flow to a region of interest, for example, a specific population of neurons close to an electrode contact. It would also be useful to target specific regions of the neurons themselves, neurites versus cell body or versus axons, in a given neuron cell population.

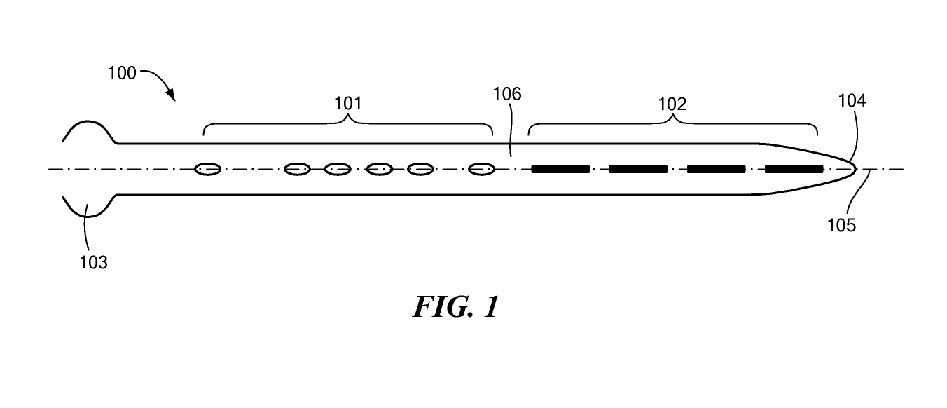

Embodiments of the present invention provide a novel approach to reach a specific neuron population at a given specific location with a cochlear implant electrode. FIG. 1 shows an example of a multi-channel cochlear implant electrode 100 according to an embodiment of the present invention where an elongate flexible electrode array 106 has a linear central axis 105, a base end 103 and an apical end 104. For electrical stimulation of nearby neural tissue, the outer surface of the electrode array 106 includes both conventional electrode contacts 101 and one or more linear electrode contacts 102 forming an elongated rectilinear surface along a line on or parallel to the linear central axis 105 of the electrode array 106. Typically, the linear electrode contacts 102 may be made of a single exposed wire around 70 microns in diameter which may be recessed below the outer surface of the electrode array 106. In some embodiments, the linear electrode contacts 102 may span a distance close to the typical amount of contact separation between two circular conventional electrode contacts 101.

If the neurites are present that form the peripheral processes of the nerve fibers (a likely situation in many etiologies of deafness), then a multi-channel implant electrode 100 having one or more linear electrode contacts 102 according to an embodiment of the present invention may be more able to activate a local region along a given length of the peripheral processes. By contrast, the shorter length conventional electrode contacts 101 are more restricted, generate more stray field, and require more current to reach the stimulation threshold. A linear electrode contact 102 at lower current can recruit a nerve fiber population along its length with action potentials that are initiated close to the contact itself where the field is highest. Increasing the current would slightly displace the electrical field forward and aside from the linear electrode contact 102, recruiting a larger population extending beyond the end of the contact and also displacing the site of spike initiation forward toward the cell body on the peripheral process. In the cochlea, a multi-channel implant electrode 100 using linear electrode contacts 102 would yield lower the stimulation threshold (low current intensity to initiate a response from the patient) and keep the action potential initiation site confined to the distal end of the neuron over a length of electrode equal to its physical length. Another consideration takes into account the apical region of the human cochlea where the upward spiraling structure possesses an ever smaller radius of curvature—the field interaction by separate electrode contacts between spiral turns can be reduced by using long linear electrode contact 102 that also allows a better and cleaner transmission of fine structure frequency information.

Lowering the threshold of stimulation also is desirable. Some electrode geometries are well-tailored to a particular morphology and so are able to offer reduced stimulation thresholds. For example, in a human cochlea the distribution of the nerve fibers becomes ever more linear and two-dimensionally planar towards the lateral wall of the scala tympani. That is, near the lateral wall and in the osseous spiral lamina, the nerves are mainly in a single plane and linearly distributed from base to apex, whereas in the modiolus, the nerve fibers are bundled together in a more three dimensional space.

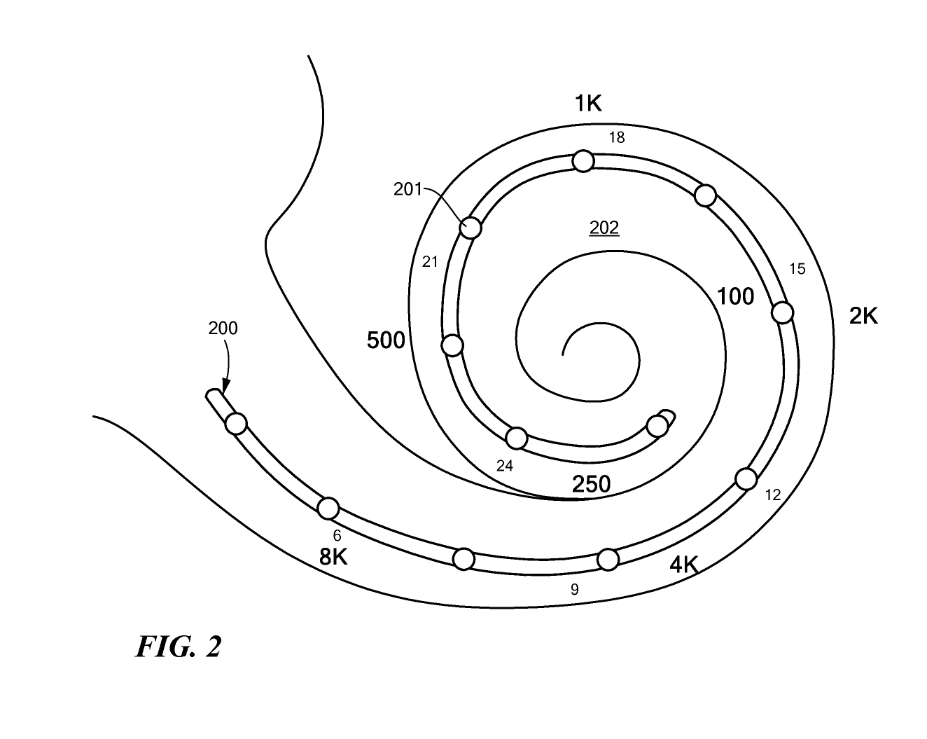

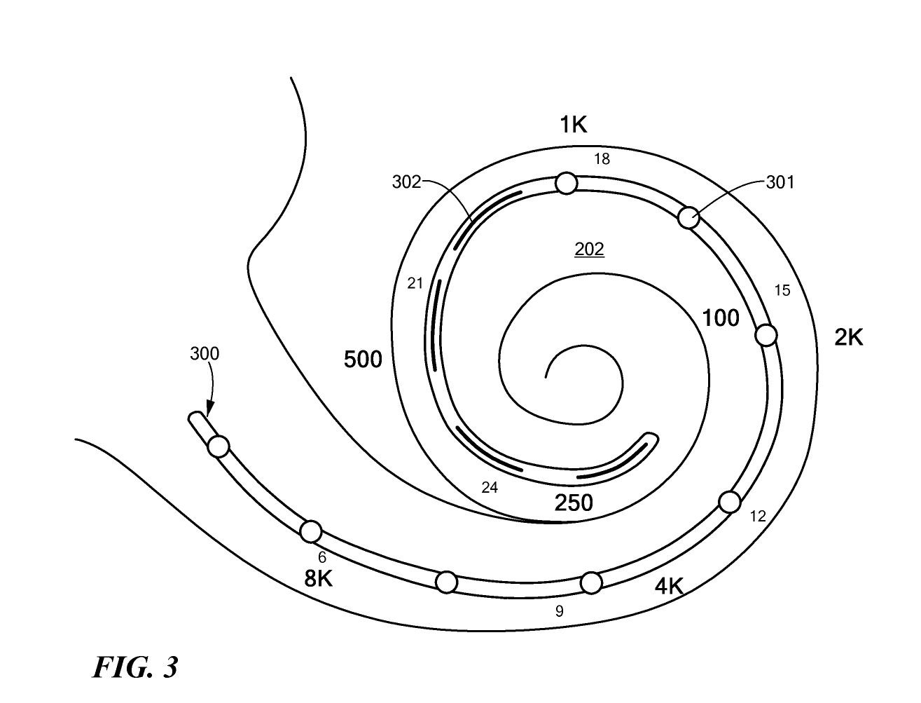

FIG. 2 shows a conventional multi-channel cochlear implant electrode 200 implanted in a human cochlea 202 according to the prior art wherein all the electrode contacts 201 have conventional circular, oval, or elliptical geometries. FIG. 3 shows a multi-channel cochlear implant electrode 300 according to an embodiment of the present invention having both conventional electrode contacts 301 and linear electrode contacts 302. In FIGS. 2 and 3, the large dark numbers around the outer circumference of the cochlea 202 show the frequency response associated with various locations within the cochlea 202 reflecting its tonotopicity. The lighter shade smaller numbers around the outer circumference of the cochlea 202 indicate the insertion depth in millimeters.

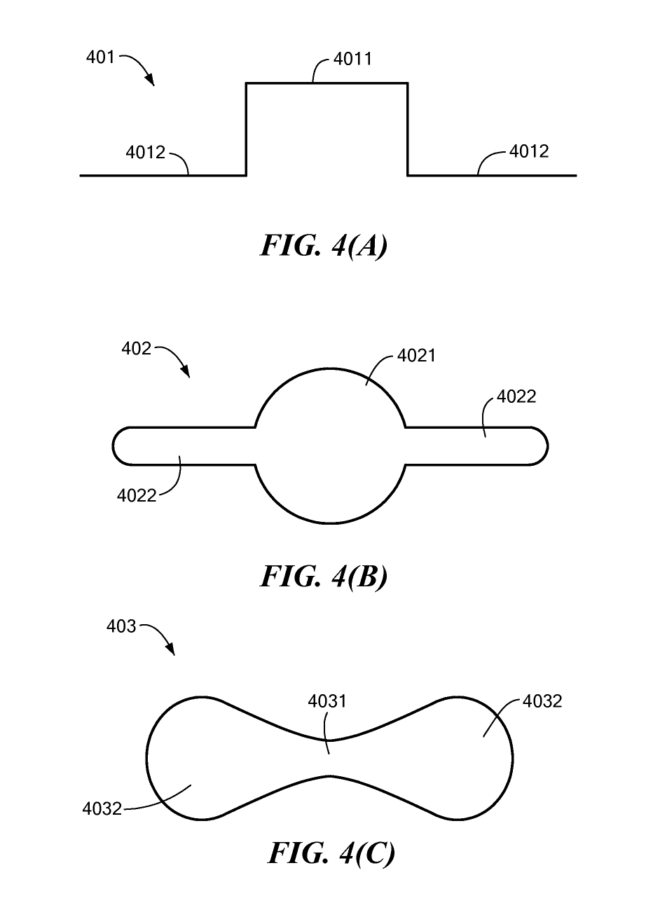

FIG. 4 A-C shows examples of specific electrode contact geometries according to specific embodiments. Specifically, FIG. 4A shows a side view of a linear electrode contact 401 wherein the center section 4011 is elevated higher than the end sections 4012, thereby increasing the intensity of the action potential created by a stimulus signal in the center section 4011. In specific embodiments, the elevated center section of the linear electrode contact 401 may be higher than the outer surface of the electrode array, flush with the surface, or recessed below the surface. FIG. 4B shows a top view of a linear electrode 402 with a rounded center segment 4021 which may or may not be at different height than the elongated ends 4022. FIG. 4C shows a linear electrode contact 403 having a rounded end segment 4032 at each end connected by a narrower elongated center section 4031.

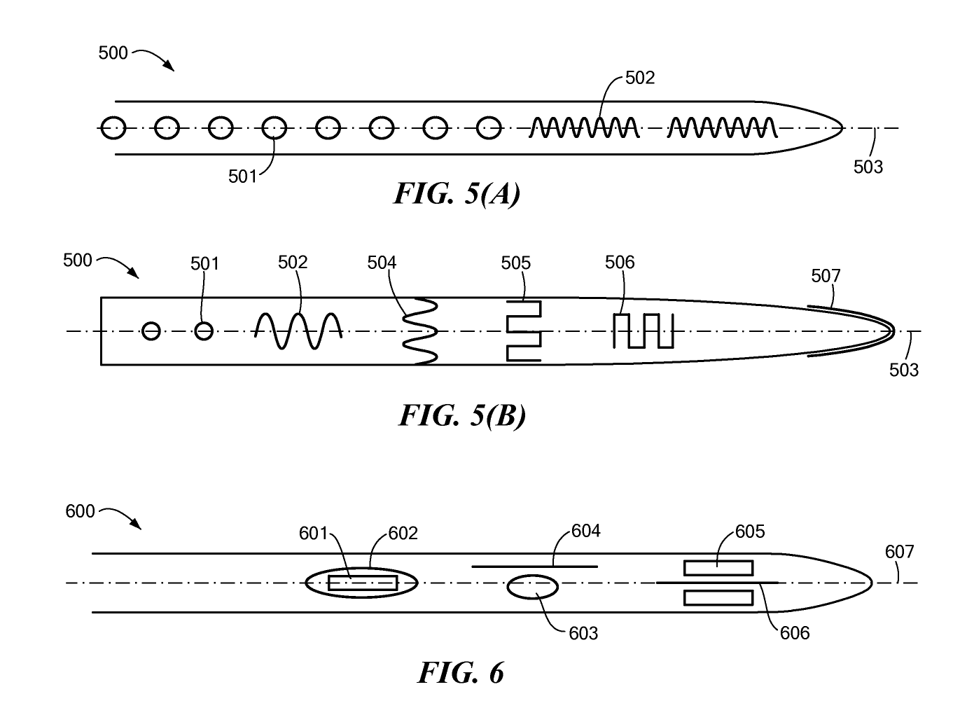

FIG. 5 A shows an example of a multi-channel implant electrode 500 according to another embodiment of the present invention having multiple wave shape linear electrode contacts 502 arranged so that overall they follow the linear central axis 503 of the electrode array. The wave shape of the linear electrode contacts 502 allows further control of the electrical field density in order to increase the current density at these locations. In other embodiments, one or more of the wave shape linear electrode contacts 502 may be located off the linear central axis 503 on a line parallel to it.

FIG. 5B shows a related embodiment where wave shape of the linear electrode contacts 504 is fixed at the circumference of the electrode 500. In specific embodiments, such a circumferential wave shape linear electrode contacts 504 can be placed completely around 360° of the electrode circumference, or the electrode contact structure could be restricted to any degree of rotation. Such an arrangement would help ensure delivery of the stimulation signal even if the body of the electrode 500 gets twisted during surgical insertion into the cochlea. Nor is the shape of such a circumferential linear electrode limited to a wave shape, but as shown in FIG. 5B, it can also have a rectangular pulse form 505 and 506. In addition, there may be an apical linear electrode contact 507 just covering the apical end of the electrode 500 to help ensure electrical stimulation of the most apical nerve tissue that the inserted electrode 500 can reach.

In some embodiments, linear electrode contacts may be differently distributed horizontally and vertically along the carrier of the electrode array. FIG. 6 shows an example of a multi-channel implant electrode 600 according to another embodiment of the present invention wherein one or more of the linear electrode contacts 604 may be distributed along different lines parallel to the linear central axis 607 of the electrode 600. In some embodiments, there may be one or more linear electrode contacts 602 arranged to enclose or partially enclose a conventional electrode contact 601.

The electrode 600 in FIG. 6 also shows an arrangement wherein there is a linear electrode contact 606 positioned substantially the same distance from an apical end of the electrode array as another conventional electrode contact 605 which in this case is actually split by the linear electrode contact 606 into two separate pieces. This configuration may be advantageous if a patient suffers from continuing nerve degradation. As long as sufficient dendrites are present, the conventional electrode contact 605 may yield better stimulation results. But when the patient's dendrites become more degraded, the linear electrode contact 606 may be more advantageous. In many embodiments, it may also be useful to simultaneously stimulate both conventional and linear electrode contacts.

A multi-channel implant electrode having one or more linear electrode contacts as discussed above offers reduced channel interaction from low to high intensity, especially in the apical region of the cochlea. In addition, the stimulation threshold for a given nerve population activation can be significantly reduced and the stimulated nerve population can be closely confined to the length of the linear electrode contact. Low intensity action potentials are initiated close to the dendrite ends of the nerve fibers reducing multiple sites of spike initiation on the same nerve fiber.

Moreover, fabrication of such an electrode and use in a patient is relatively easy. There can be a reduced amount of material on the electrode array carrier. For example, a linear electrode contact may typically be formed from a 70 micron diameter wire 1.5 mm in length which is half embedded in the carrier material. By comparison, a conventional electrode contact might be 60×800×25 microns. For a 0.14 mm² contact area, an exposed conventional electrode contact that is 300 microns in diameter can be replaced by a half embedded linear electrode contact wire 60 microns in diameter and 1.4 mm long having the same surface area.

Although various exemplary embodiments of the invention have been disclosed, it should be apparent to those skilled in the art that various changes and modifications can be made which will achieve some of the advantages of the invention without departing from the true scope of the invention.

What is claimed is:

1. A cochlear implant electrode comprising:
an elongate flexible electrode array having a linear central axis and an outer surface with a plurality of electrode contacts for electrical stimulation of nearby neural tissue;
wherein at least one of the electrode contacts is a linear electrode contact forming an elongated rectilinear surface along a line parallel to the linear central axis of the electrode array.

2. A cochlear implant electrode according to claim 1, wherein a plurality of electrode contacts are linear electrode contacts.

3. A cochlear implant electrode wherein the plurality of linear electrode contacts are distributed along different lines parallel to the linear central axis of the electrode array.

4. A cochlear implant electrode according to claim 1, wherein the at least one linear electrode contact is located towards an apical end of the electrode array.

5. A cochlear implant electrode according to claim 1, wherein at least a portion of the linear electrode contact is elevated above the outer surface of the electrode array.

6. A cochlear implant electrode according to claim 5, wherein the elevated portion is substantially centered within the at least one linear electrode contact.

7. A cochlear implant electrode according to claim 1, wherein at least a portion of the linear electrode contact is recessed below the surface of electrode array.

8. A cochlear implant electrode according to claim 1, wherein the at least one linear electrode contact includes a rounded center segment.

9. A cochlear implant electrode according to claim 1, wherein the at least one linear electrode contact includes a rounded end segment.

10. A cochlear implant electrode according to claim 9, wherein the at least one linear electrode contact includes a rounded end segment at each end.

11. A cochlear implant electrode according to claim 1, wherein the at least one linear electrode contact includes a plurality of wave shape segments each following a line off the linear central axis arranged so that overall the at least one linear electrode contact follows a line parallel to the linear central axis of the electrode array.

12. A cochlear implant electrode according to claim 1, wherein at least one linear electrode contact is positioned substantially the same distance from an apical end of the electrode array as another electrode contact.

13. A cochlear implant electrode according to claim 1, wherein the at least one linear electrode contact is located around a circumferential outer surface of the electrode array.

14. A cochlear implant electrode according to claim 1, wherein the at least one linear electrode contact is located at an apical end outer surface of the electrode array.
